(19) United States
(12) Patent Application Publication
(10) Pub. No.: US 2024/0260740 A1
(43) Pub. Date: Aug. 8, 2024

(54) COSMETIC DEVICE

(71) Applicant: LG ELECTRONICS INC., Seoul (KR)

(72) Inventors: Woohyun KIM, Seoul (KR); Hyoungjun KIM, Seoul (KR); Yonggeun JIN, Seoul (KR)

(73) Assignee: LG ELECTRONICS INC., Seoul (KR)

(21) Appl. No.: 18/566,984

(22) PCT Filed: Jul. 9, 2021
(86) PCT No.: PCT/KR2021/008791

§ 371 Date: Dec. 4, 2023

(30) Foreign Application Priority Data
Jun. 25, 2021 (KR) 10-2021-0083441

(51) Int. Cl. A46B 15/00; A46B 13/02; A61N 1/04

(52) U.S. Cl. CPC A46B 15/0055; A46B 13/023; A61N 1/0428; A46B 2200/1006; A46B 2200/1053

(57) ABSTRACT

A cosmetic device comprises: a case within which a space is formed; an absorption-promoting head which is disposed at one of both side surfaces of the case; an ultrasonic head and a brush unit which are disposed at the other of both side surfaces of the case; a vibration motor which is accommodated in the space to vibrate at least one of the ultrasonic head and the brush unit; a controller which is accommodated in the space; and an iontophoresis electrode which is closer to the brush unit than to the absorption-promoting head and a part of which is exposed out of the case.

### TECHNICAL FIELD

The present invention relates to a cosmetic device.

### BACKGROUND ART

An example of a cosmetic device includes a skin cleansing device that removes or cleans makeup residues, sebum, oil, and other wastes from human skin through vibration.

Examples of such a skin cleaning device is disclosed in Korean Patent Publication No. 10-2259865 B1 (published on Jun. 3, 2021), and the skin cleaning device includes: a body case provided in a semi-disc shape to be held in a state of being in close contact with the user's palm and having one side that is flat and the other side that is rounded; a rubber brush mounted on the flat one surface of the body case, having a through-hole defined in a center thereof, and provided with a plurality of protrusions made of a rubber material on an outer surface thereof; a vibration motor mounted inside the body case to allow the rubber brush to rotate by supplying power; a battery mounted inside the body case and configured to supply power to various devices including the vibration motor; and a power control board mounted inside the body case and connected to the vibration motor and a battery to control power supply to the vibration motor.

Another example of the cosmetic device include an iontophoresis device that is capable of improving absorption of active ingredients such as cosmetics, and examples of the iontophoresis include an iontophoresis device disclosed in Korean Patent Registration No. 10-0730582 B1 (published on Jun. 20, 2007), which includes a plurality of electrodes installed to be built in a mask or patch attached to the user's skin, and a chip module for iontophoresis, which is configured to be attachable and detachable at a predetermined position on the mask or patch and is electrically connected to the electrodes.

### DISCLOSURE OF THE INVENTION

### Technical Problem

An object of the present invention is to provide a cosmetic device having a cleaning function and an absorption-promoting function of cosmetics to provide an easy-to-use and high portability.

### Technical Solution

A cosmetic device according to the present embodiment includes: a case in which a space is defined; an absorption-promoting head disposed on one surface of both side surfaces of the case; an ultrasonic head and a brush unit, which are disposed on the other surface of both the side surfaces of the case; a vibration motor accommodated in the space to vibrate at least one of the ultrasonic head or the brush unit; a controller accommodated in the space; and an iontophoresis electrode which is closer to the brush unit than to the absorption-promoting head and of which a portion is exposed to the outside of the case.

The controller may be configured to perform: an absorption-promoting mode in which the absorption-promoting head and the iontophoresis are electrically connected to each other; and a cleansing mode in which the ultrasonic head and the vibration motor operate.

The case may include: a center case of which an outer surface is concavely recessed; a first side case coupled to one side of the center case; and a second case coupled to the other side of the center case, wherein the second side case may have an opening through which the ultrasonic head passes.

The center case may have a horizontal width that gradually increases downward.

The cosmetic device may further include an inner sealing member.

The inner sealing member may be disposed along an inner circumferential surface of the center case.

The cosmetic device may further include: a first side case sealing member and a second side case sealing member.

The first side case sealing member may be disposed on the first side case to seal a gap between the first side case and the center case.

The second side case sealing member may be disposed on the second side case to seal a gap between the second side case and the center case.

A distance between an upper end of the absorption-promoting head and an upper end of the brush unit may be less than a distance between a lower end of the absorption-promoting head and a lower end of the brush head.

The cosmetic device may further include a head ultrasonic vibrator disposed on the absorption-promoting head.

The absorption-promoting head may have a cam shape.

A distance between an upper end of the brush unit and the ultrasonic head may be greater than a distance between a lower end of the brush unit and the ultrasonic head.

The brush head may be configured to cover a portion of the iontophoresis electrode outside the case.

The iontophoresis electrode may include a hidden body and an exposed body.

The hidden body may be hidden by the brush unit between the case and the brush unit.

The exposed body may extend from the hidden body and be exposed to the outside of the case.

The ultrasonic head may include an inner head and an ultrasonic vibrator.

The inner head may be disposed inside the brush unit. An accommodation space may be defined in the inner head.

The ultrasonic vibrator may be accommodated in the accommodation space.

The cosmetic device may further include an inner head sealing member.

The inner head sealing member may be disposed on an outer circumference of the inner head to seal a gap between the inner head and the case.

The controller may include a main PCB and a sub PCB connected to the main PCB.

A spring may be disposed between the sub PCB and the iontophoresis electrode. The spring may electrically connect the iontophoresis electrode to the sub PCB.

### Advantageous Effects

According to the embodiment of the present invention, the cleaning and absorption promotion of the cosmetics may be realized through one cosmetic device to improve the convenience of use and realize compactification and easy portability during the outdoor activates.

In addition, the one surface of the cosmetic device may have the absorption-promoting function, and the other surface of the cosmetic device may have the cleaning function, and thus, both the surfaces of the cosmetic device may be used.

In addition, in the state in which the user's hand touches the iontophoresis electrode adjacent to the brush unit, the absorption-promoting head may approach the user's face to improve the convenience in use.

In addition, the penetration of the moisture into the case space may be minimized to improve the reliability.

In addition, there may be advantage in that the upper portion of the brush unit is easy to enter the local areas such as next to the nose or next to the eyes, and thus, the fine cleaning is easy.

In addition, since the brush unit covers the portion of the iontophoresis electrode outside the case, the separate cover member for covering the iontophoresis electrode may be unnecessary, and the number of components may be minimized.

In addition, the inner head sealing member may seal the gap between the ultrasonic head and the case.

In addition, since the iontophoresis electrode is connected to the sub PCB by the spring, the connection state of the iontophoresis electrode may be maintained in high reliability.

### BRIEF DESCRIPTION OF THE DRAWINGS

FIG. 1 is a front view of a cosmetic device according to the present embodiment,

FIG. 2 is a perspective view illustrating an absorption-promoting head of the cosmetic device according to the present embodiment,

FIG. 3 is a perspective view illustrating a brush unit of the cosmetic device according to the present embodiment,

FIG. 4 is a side view illustrating the brush unit of the cosmetic device according to the present embodiment,

FIG. 5 is an exploded perspective view of the cosmetic device according to the present embodiment,

FIG. 6 is a perspective view of the cosmetic device in which the absorption-promoting head and the brush unit are separated from each other according to the present embodiment,

FIG. 7 is a longitudinal cross-sectional view of the cosmetic device according to the present embodiment, and

FIG. 8 is a longitudinal cross-sectional view of the cosmetic device according to the present embodiment.

### MODE FOR CARRYING OUT THE INVENTION

Hereinafter, detailed embodiments will be described in detail with reference to the accompanying drawings.

Hereinafter, embodiments of the present disclosure are described in more detail with reference to accompanying drawings and regardless of the drawings symbols, same or similar components are assigned with the same reference numerals and thus overlapping descriptions for those are omitted. The suffixes “module” and “unit” for components used in the description below are assigned or mixed in consideration of easiness in writing the specification and do not have distinctive meanings or roles by themselves.

In the following description, detailed descriptions of well-known functions or constructions will be omitted since they would obscure the disclosure in unnecessary detail. However, this does not limit this specification within specific embodiments and it should be understood that the present disclosure covers all the modifications, equivalents, and replacements within the idea and technical scope of this specification.

It will be understood that the terms “first” and “second” are used herein to describe various components but these components should not be limited by these terms. The terms are only used to distinguish one component from other components.

In this disclosure below, when one part (or element, device, etc.) is referred to as being ‘connected’ or ‘disposed’ to another part (or element, device, etc.), it should be understood that the former can be ‘directly connected’ or ‘directly disposed’ to the latter via an intervening part (or element, device, etc.).

The terms of a singular form may include plural forms unless referred to the contrary.

In this application, the terms “comprises” or “having” are intended to indicate that there is a feature, number, step, operation, component, part, or combination thereof described in the specification, and one or more other features. It is to be understood that the present invention does not exclude the possibility of the presence or the addition of numbers, steps, operations, components, components, or a combination thereof.

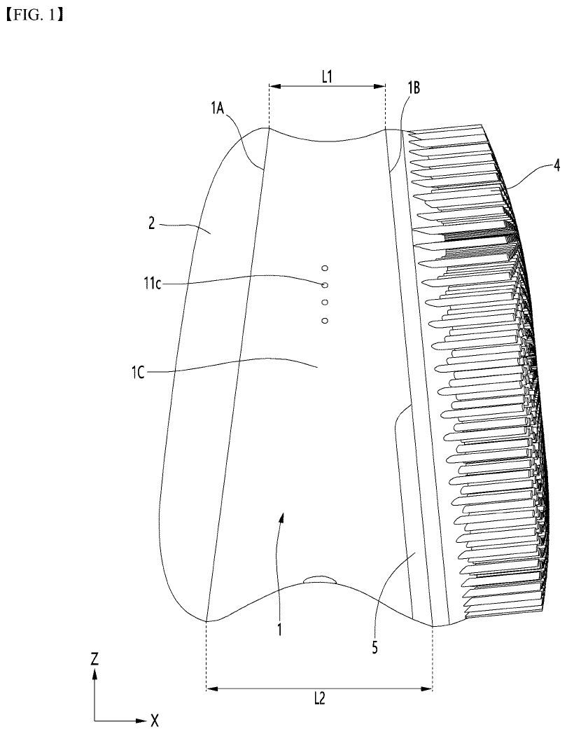

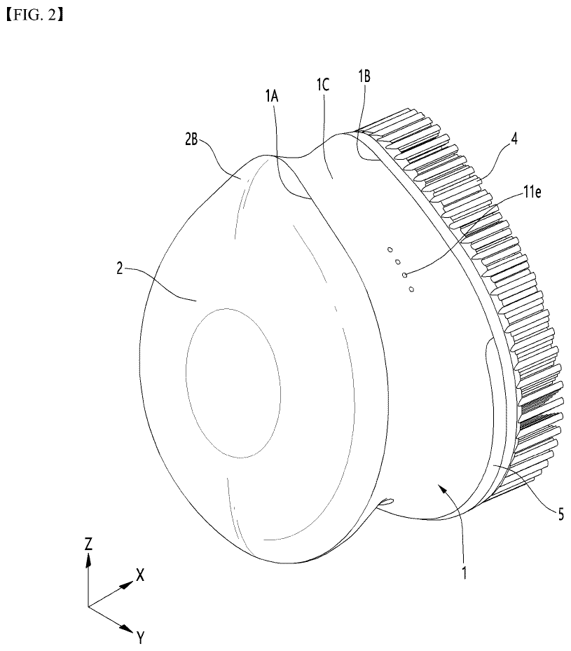

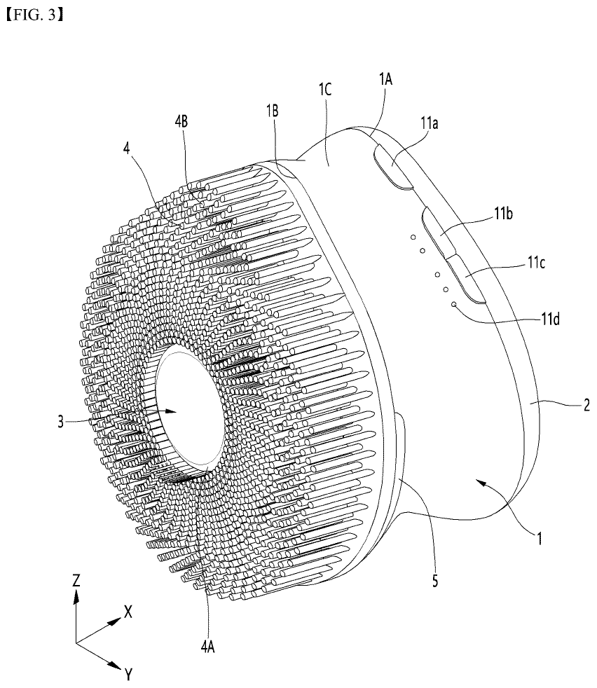

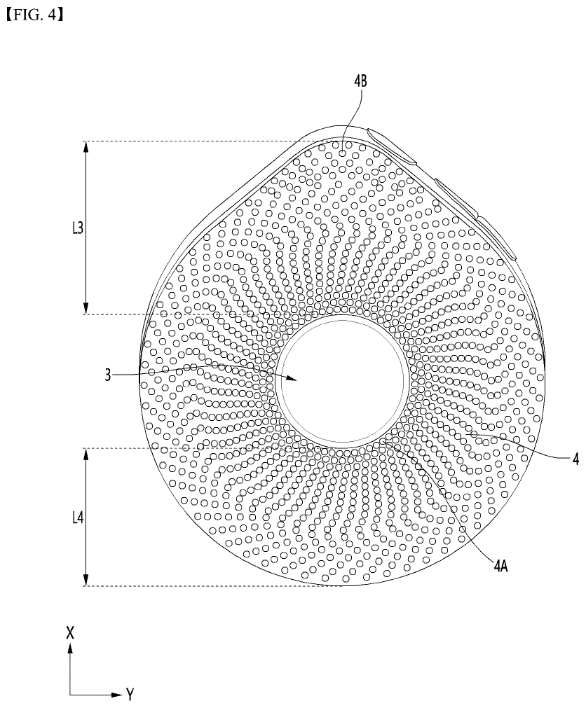

FIG. 1 is a front view of a cosmetic device according to the present embodiment, FIG. 2 is a perspective view illustrating an absorption-promoting head of the cosmetic device according to the present embodiment, FIG. 3 is a perspective view illustrating a brush unit of the cosmetic device according to the present embodiment, and FIG. 4 is a side view illustrating the brush unit of the cosmetic device according to the present embodiment.

A cosmetic device may include a case 1, an absorption-promoting head 2, an ultrasonic head 3, a brush unit 4, and an iontophoresis electrode 5.

The case 1 may be disposed between the absorption-promoting head 2 and the brush unit 4.

The case 1 may define an outer appearance of a central side of the cosmetic device, and the absorption-promoting head 2 and the brush unit 4 may be spaced apart from each other in a left and right direction X.

The case 1 may include one surface 1A, the other surface 1B, and an outer surface 1C.

The outer surface 1C may be provided to connect an edge of the one surface 1A to an edge of the other surface 1B, and the outer surface 1C may be provided to be recessed.

When holding the outer surface 1C, a user may easily hold the cosmetic device by his/her hands, and a portion of the case 1, which is disposed between the absorption-promoting head 2 and the brush unit 4 may function as a handle.

When using the absorption-promoting head 2 in the cosmetic device, the user may hold a portion of the case 1 using the his/her fingers while wrapping the brush unit 4 using the his/her hands, and the outer surface 1C of the case 1 may be recessed to improve grip feeling and minimize a phenomenon in which the user's hand slip during use of the cosmetic device.

When using the ultrasonic head 3 and brush unit 4 in the cosmetic device, the user may hold a portion of the case 1 using the his/her fingers while wrapping the absorption-promoting head 2 using the his/her hands, and the outer surface 1C of the case 1 may be recessed to improve the grip feeling and minimize the phenomenon in which the user's hand slip during use of the cosmetic device.

As illustrated in FIG. 3, at least one input unit 11a, 11b, and 11c and an indicator 11d displaying information about the cosmetic device may be disposed in the case 1.

An example of the input units 11a, 11b, and 11c may be configured as buttons.

At least one input units 11a, 11b, 11c may include a power input unit 11a for inputting power, a mode input unit 11b for selecting a mode, and a level input unit 11c for selecting a level.

The cosmetic device may include an absorption-promoting mode (first mode or iontophoresis mode) and a cleansing mode (second mode).

The absorption-promoting mode may be a mode in which the absorption-promoting head 2 that is in contact with the skin promotes absorption of liquid substances such as cosmetics. The cleansing mode may be a mode in which the ultrasonic head 3 and the brush unit 4 that is in contact with the skin cleanse the skin.

The first mode and the second mode may be selected depending on the number of times of pressing of the mode input unit 11b.

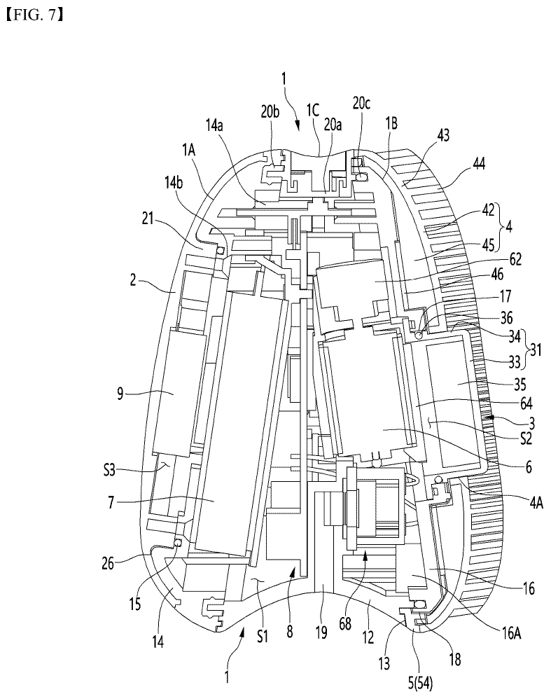

As illustrated in FIG. 2, a battery indicator 11e may be disposed in the case 1 to display a charging state of the battery 7 (see FIG. 7), which will be described later.

The indicator 11d and the battery indicator 11e may be disposed in a recessed region of the outer surface 1C of the case 1.

The case 1 may have a shape that expands toward the bottom, as illustrated in FIG. 1. A width of the case 1 in the left and right directions X may increase toward the bottom. Both surfaces 1A and 1B of the case 1 may be inclined surfaces that are inclined with respect to a vertical line, and the inclined surfaces may have an acute inclined angle with the vertical line.

A distance L1 between an upper end of the absorption-promoting head 2 and an upper end of the brush unit 4 may be less than a distance L2 between a lower end of the absorption-promoting head 2 and a lower end of the brush unit 4.

The absorption-promoting head 2 and the brush unit 4 may be arranged to be inclined at a predetermined angle or may be arranged to have an acute inclination angle.

The absorption-promoting head 2 may be disposed on the one surface 1A of both the surfaces 1A and 1B of the case 1. On the other hand, the ultrasonic head 3 (see FIG. 3) and the brush unit 4 may be disposed on the other surface 1B of both the surfaces 1A and 1B of the case 1.

The absorption-promoting head 2 and the ultrasonic head 3 may be disposed on opposite surfaces of the one surface 1A of the case 1 and the other surface 1B of the case 1, respectively. Here, the one surface 1A of the case 1 and the other surface 1B of the case 1 may not in contact with each other.

Here, when one surface 1A is a left surface of the left and right surfaces of the case 1, and the other surface 1B may be the right surface of the left and right surfaces of the case 1. Conversely, if the one surface 1A is the right surface of the case 1, the other surface 1B may be the left surface of the case 1.

An outer circumference of the absorption-promoting head 2 may be provided as a whole in a cam shape or a water drop shape, and a portion similar to the cam nose 2B (see FIG. 2) may be easily entered into a narrow region such as next to the user's nose.

The ultrasonic head 3 may be disposed at a central portion of the other surface 1B of the case 1.

The brush unit 4 may be disposed close to the edge of the other surface 1B of the case 1.

As illustrated in FIGS. 3 and 4, an opening 4A surrounding an outer circumference of the ultrasonic head 3 may be defined in a center of the brush unit 4, and the ultrasonic head 3 may be disposed inside the opening 4A.

The ultrasonic head 3 may be exposed to the outside through the opening 4A, and when the user places the brush unit 4 on the skin, the ultrasonic head 3 may be in contact with the user's skin together with the brush unit 4.

An outer circumference of the brush unit 4 may be provided as a whole in the cam shape or the water drop shape, and a portion similar to the cam nose 4B may be easily entered into a narrow region such as next to the user's nose.

As illustrated in FIG. 4, the portion 4B of the brush unit 4, which has the shape similar to the cam nose, may be disposed on an upper portion of the brush unit 4. A distance L3 between an upper end of the brush unit 4 and the ultrasonic head 3 may be greater than a distance L4 between a lower end of the brush unit 4 and the ultrasonic head 3.

The iontophoresis electrode 5 may be an electrode that is in contact with the user's hand when using the absorption-promoting head 2. The iontophoresis electrode 5 may be disposed closer to the brush unit 4 of the absorption-promoting head 2 and the brush unit 4.

When using the absorption-promoting head 2, the iontophoresis electrode 5 may be preferably disposed at a position at which the user's skin, for example, is in easily contact with the hand. The iontophoresis electrode 5 may be closer to the brush unit 4 than the absorption-promoting head 2. The iontophoresis electrode 5 may be spaced apart from the absorption-promoting head 2 and may be in contact with the brush unit 4.

An example of the iontophoresis electrode 5 may be a zinc alloy.

A portion of the iontophoresis electrode 5 may be exposed to the outside of the case 1. When using the absorption-promoting head 2, the user may wrap the brush unit 4 and the surroundings of the brush unit 4 (e.g., a portion of the case 1 and the iontophoresis electrode 5) by using the his/her hand. Here, a portion of the user's fingers may be in contact with the iontophoresis electrode 5.

When the user's hand is in contact with the iontophoresis electrode 5, and the absorption-promoting head 2 is in contact with the user's face, etc., the absorption-promoting head 2 and the iontophoresis electrode 5 may be electrically connected by the user.

The absorption-promoting head 2 may have a −polarity, and the iontophoresis electrode 5 may have a + polarity opposite to that of the absorption-promoting head 2, and negative ions may output from the absorption-promoting head 2.

The negative ions output from the absorption-promoting head 2 may push negative charges in cosmetics into the skin, and the cosmetics may be more quickly penetrated into the skin by the negative ions output from the absorption-promoting head 2.

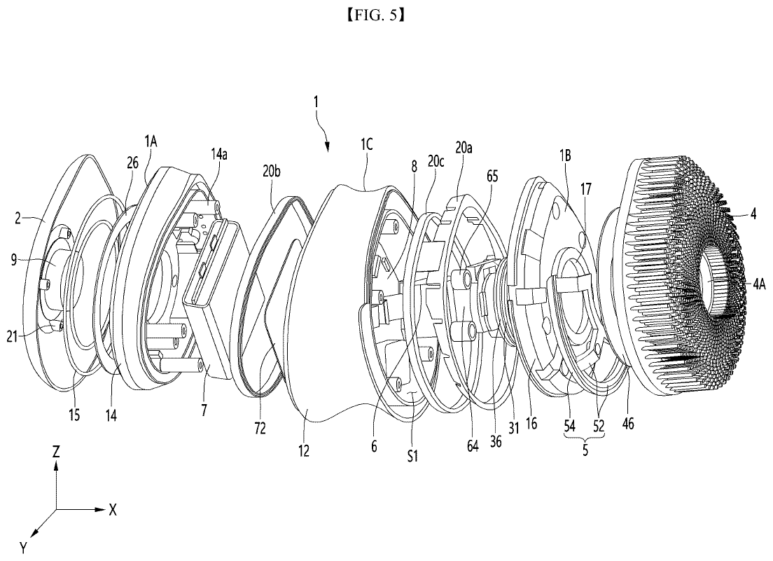

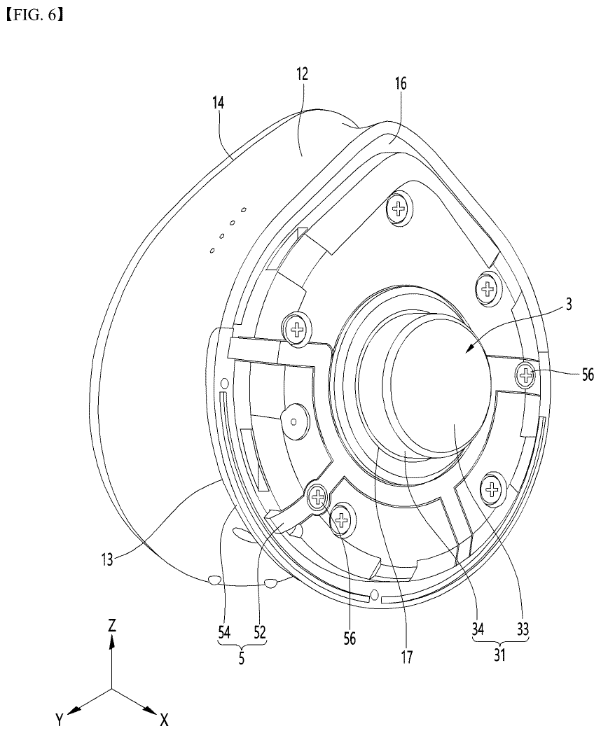

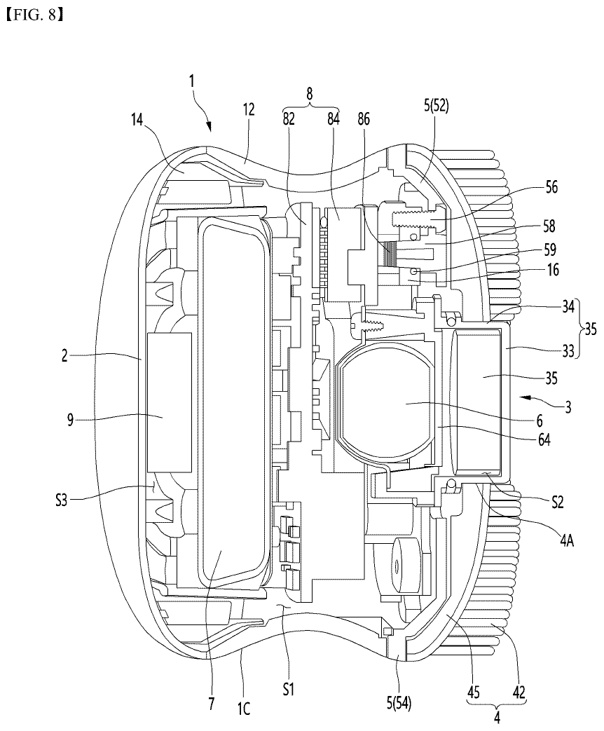

FIG. 5 is an exploded perspective view of the cosmetic device according to the present embodiment, FIG. 6 is a perspective view of the cosmetic device in which the absorption-promoting head and the brush unit are separated from each other according to the present embodiment, FIG. 7 is a longitudinal cross-sectional view of the cosmetic device according to the present embodiment, and FIG. 8 is a longitudinal cross-sectional view of the cosmetic device according to the present embodiment.

As illustrated in FIG. 5, the cosmetic device may further include a vibration motor 6, a battery 7, and a controller 8.

A space S1 may be defined inside the case 1. The case 1 may be provided as a combination of a plurality of case members, and the space S1 may be defined by the plurality of case members.

The case 1 may include a center case 12, a first side case 14, and a second side case 16.

The center case 12 may have a space S1 defined therein, and both left and right surfaces may be opened. The center case 12 may be opened in a left and right direction X. An outer surface 1C of the center case 12 may be concavely recessed.

The center case 12 may have a shape that expands toward the bottom. A width of the center case 12 in the left and right direction X may increase toward the bottom.

The first side case 14 may define the space S1 together with the center case 12.

The first side case 14 may be coupled to one side of the center case 12, the first side case 14 may cover one surface of the center case 12, and the outer surface may be defined as the one surface 1A of the case 1.

The first side case 14 may be provided with a cable through-part through which a cable passes.

The first side case 14 may be coupled to the center case 12 through a coupling member such as a screw, and the coupling member such as the screw may be coupled to the first side case 14, and a coupling boss 14a (see FIGS. 5 and 7) coupled to the center case 12 may be provided.

The first side case 14 may be coupled to the absorption-promoting head 2 through the coupling member such as the screw, and the first side case 14 may have a coupling member through-hole 14b through which the coupling member such as the screw passes.

As illustrated in FIGS. 5 and 7, the cosmetic device may further include a head sealing member 15. The head sealing member 15 may seal a gap between the absorption-promoting head 2 and the case 1.

The head sealing member 15 may be disposed on the first side case 14. The head sealing member 15 may be disposed on a surface of the first side case 14, which faces the absorption-promoting head 2, and may be disposed between the absorption-promoting head 2 and the first side case 14.

An example of the head sealing member 15 may be a waterproof O-ring that seals a gap between the absorption-promoting head 2 and the first side case 14.

The head sealing member 15 may prevent moisture from being penetrated between the absorption-promoting head 2 and the case 1.

The second side case 16 may define a space S1 together with the center case 12.

The second side case 16 may be coupled to the other side of the center case 12, the second side case 16 may cover the other side of the center case 12, and an outer surface of the second side case 16 may be defined as the other surface 1B of the case 1.

The second side case 16 may be provided with an opening 17 through which the ultrasonic head 3 passes. The opening 17 may be opened approximately in the left and right direction.

The second side case 16 may be coupled to the center case 12 through a coupling member such as a screw, and the coupling member such as the screw may be coupled to the second side case 16, and a coupling boss 16A coupled to the center case 12 may be provided.

The cosmetic device may include a sealing member that prevents external moisture (e.g., cosmetics or foam, etc.) from being penetrated into the space S1 of the case 1.

As illustrated in FIGS. 5 and 7, the cosmetic device may further include an inner sealing member 20a.

The inner sealing member 20a may be disposed along an inner circumferential surface of the center case 12. An overall shape of the inner sealing member 20a may be ring-shaped. An example of the inner sealing member 20a may be waterproof silicone.

As illustrated in FIGS. 5 and 7, the cosmetic device may further include a first side case sealing member 20b and a second side case sealing member 20b.

The first side case sealing member 20b may be disposed on the first side case 14 to seal a gap between the first side case 14 and the center case 12. An example of the first side case sealing member 20b may be waterproof silicone.

The second side case sealing member 20c may be disposed on the second side case 16 to seal a gap between the second side case 16 and the center case 12. An example of the second side case sealing member 20c may be waterproof silicone.

The absorption-promoting head 2 may be disposed to cover the one surface 1A of the case 1. The absorption-promoting head 2 may be provided in a convex shape in an opposite direction of the case 1.

The absorption-promoting head 2 may be a SUS head, and an example of the absorption-promoting head 2 may be SUS 416L.

The absorption-promoting head 2 may be coupled to the first side case 14 using the coupling member such as the screw. A coupling part 21 (see FIGS. 5 and 7) coupled to the first side case 16 may be disposed on the absorption-promoting head 2. The coupling member such as the screw may pass through the coupling member through-hole 14b of the first side case 14 and be coupled to the coupling part 21.

A waterproof member 26 (see FIGS. 5 and 7) may be disposed between the absorption-promoting head 2 and the case 1.

An example of the waterproof member 26 may be a double-sided tape for waterproofing, and the absorption-promoting head 2 may be disposed between an inner surface of and an outer surface 1A of the first side case 14. The waterproof member 26 may be formed in an arc shape, a ring shape, or a hollow disk shape. The waterproof member 26 may fix the absorption-promoting head 2 to the case 1 to seal a gap between the absorption-promoting head 2 and the case 1.

As illustrated in FIG. 7, the ultrasonic head 3 may include an inner head 31 and an ultrasonic vibrator 35.

The inner head 31 may have an accommodation space S2 defined therein and may be disposed inside the brush unit 4.

The inner head 31 may be a SUS head, and an example of the inner head 31 may be SUS 416L.

The inner head 31 may include a head 33 exposed to the outside, and a hollow body 34 provided in the head 33 to define the accommodation space S2.

The head 33 may be in contact with the user's skin and may supply ultrasonic waves to the user's skin.

The hollow body 34 may surround the ultrasonic vibrator 35 and protect the ultrasonic vibrator 35.

An outer surface of the hollow body 34 may be in contact with the opening 4A of the brush unit 4.

The ultrasonic vibrator 35 may be accommodated in the accommodation space S2, and the ultrasonic vibrator 35 may be protected by the inner head 31.

The ultrasonic vibrator 35 may include an inner head 31 and an ultrasonic module.

The cosmetic device may further include an inner head sealing member 36 (see FIGS. 5 and 7). The inner head sealing member 36 may seal a gap between the inner head 31 and the case 1. The inner head sealing member 36 may be disposed on an outer circumference of the inner head 31. The inner head sealing member 36 may be disposed between the opening 17 of the second side case 16 and the hollow body 34 of the inner head. An example of the inner head sealing member 36 may be a waterproof O-ring. The inner head sealing member 36 may prevent moisture from being penetrated between the inner head 31 and the case 1.

As illustrated in FIG. 7, the brush unit 4 may include a silicone brush 42 and a brush supporter 45.

The silicone brush 42 may include a body 43 covering the brush supporter 45 and a brush 44 that protrudes from one surface of the body 43 and is bent.

The brush supporter 45 may be disposed between the silicone brush 42 and the second side case 16 and may support the silicone brush 42.

When the brush unit 4 is mounted, the brush unit 4 may cover a portion 52 of the iontophoresis electrode 5 outside the case 1.

A waterproof member 46 (see FIGS. 5 and 7) may be disposed between the brush unit 4 and the case 1.

An example of the waterproof member 46 may be a double-sided tape for waterproofing, and the waterproof member 46 may be disposed between an inner surface of the brush supporter 45 and the outer surface 1B of the second side case 16. The waterproof member 46 may be formed in an arc shape, a ring shape, or a hollow disk shape. The waterproof member 46 may fix the brush unit 4 to the case 1 and seal a gap between the brush unit 4 and the case 1.

The iontophoresis electrode 5 may include a hidden body 52 and an exposed body 54, as illustrated in FIG. 6.

The hidden body 52 may be a portion, which is hidden by the brush unit 4 when the brush unit 4 is mounted, and also may be defined as a portion of the iontophoresis electrodes 5, which is disposed between the case 1 and the brush unit 4.

The hidden body 52 may be coupled to the case 1, particularly the second side case 16, through the coupling member 56. A through-hole through which the coupling member 56 such as the screw passes may be defined in the hidden body 52, and the coupling member 56 may pass through the through-hole of the hidden body 52 and then be coupled to the coupling part provided on the second side case 16.

The exposed body 54 may extend to protrude from the hidden body 52.

The exposed body 54 may be a portion that is not hidden by the brush unit 4 when the brush unit 4 is installed and also may be a portion exposed to the outside of the case 1 through the case 1 and the brush unit 4.

The case 1, particularly the center case 12, may be provided with an electrode accommodation part 13 (see FIGS. 6 and 7) in which the exposed body 54 is accommodated.

A pressing body 18 (see FIG. 7) that is capable of pressing the exposed body 54 to the electrode accommodation part 13 may be provided in the case 1, particularly the second side case 16.

That is, the hidden body 52 of the iontophoresis electrode 5 may be fixed to the second side case 16 through the coupling member 56, and the exposed body 54 of the iontophoresis electrode 5 may be fixed to be exposed to the outside between the center case 12 and the second side case 16.

The vibration motor 6 (see FIGS. 7 and 8) may be accommodated in the space S1. The vibration motor 6 may vibrate at least one of the ultrasonic head 3 or the brush unit 4.

The vibration motor 6 may be accommodated in the space S1 so as to be closer to the brush unit 4 of the absorption-promoting head 2 and the brush unit 4. The vibration motor 6 may be accommodated in the space S1 so as to be closer to the ultrasonic head 3 of the absorption-promoting head 2 and the ultrasonic head 3.

A weight 62 may be connected to a rotation axis of the vibration motor 6.

The cosmetic device may further include a motor mounter 64. The motor mounter 64 may surround an outer circumference of the vibration motor 6. The motor mounter 64 may be coupled to the case 1.

The motor mounter 64 may be provided with the coupling part 65 (see FIG. 5) that is coupled to the case 1 through the coupling member such as the screw.

As illustrated in FIG. 7, the motor mounter 64 may be disposed to be in contact with the ultrasonic head 3, especially the inner head 31, and vibration generated by the vibration motor 6 may be transmitted to the inner head 31 through the motor mounter 64. The vibration transmitted to the inner head 31 may be transmitted to the brush unit 4 through the opening 4A.

A speaker unit 66 capable of transmitting information about the cosmetic device through voice may be disposed around the motor mounter 64. The speaker unit 66 may be disposed to overlap the vibration motor 6 in a vertical direction Z.

A speaker hole 19 (see FIG. 7) that is capable of transmitting the sound of the speaker unit 66 to the outside may be defined in the case 1, especially the center case 12.

The battery 7 may be accommodated in the space S1, as illustrated in FIG. 7. The battery 7 may be accommodated between the first side case 4 and the center case 4.

A battery insulating sheet 72 (see FIG. 5) may be disposed between the battery 7 and the controller 8. The battery 7 may be insulated by the battery insulating sheet 72.

The controller 8 may be accommodated in the space S1. The controller 8 may be disposed inside the center case 12. The controller 8 may divide the space S1 into left and right sides. The space S may include a battery space in which the battery 7 is accommodated, and a vibration motor space in which the vibration motor 6 and the speaker unit 66 are accommodated.

The controller 8 may implement the absorption-promoting mode and the cleansing mode. The absorption-promoting mode and cleansing mode may be selectively implemented.

The absorption-promoting mode may be a mode in which the absorption-promoting head 2 and the iontophoresis electrode 5 are electrically connected to generate galvanic current in the absorption-promoting head 2.

The cleansing mode may be a mode in which the ultrasonic head 3 and the vibration motor 6 operate. In the cleansing mode, the ultrasonic vibrator 35 and the vibration motor 6 may be turned on.

The cosmetic device may further include a head ultrasonic vibrator 9 (see FIG. 7).

The head ultrasonic vibrator 9 may be disposed on the absorption-promoting head 2.

The head ultrasonic vibrator 9 may be disposed between the absorption-promoting head 2 and the battery 7. The head ultrasonic vibrator 9 may be provided to be smaller than the absorption-promoting head 2 and may be accommodated in the inner space S3 of the absorption-promoting head 2.

The controller 8 may be provided as a combination of multiple PCBs.

The controller 8 may include a main PCB 82 (see FIGS. 7 and 8) and a sub PCB 84 (see FIG. 8).

The main PCB 82 may be accommodated in a space defined inside the center case 12 and may be protected by the center case 82. As illustrated in FIG. 7, the main PCB 82 may be disposed to be elongated in the vertical direction Z inside the center case 12.

The sub PCB 84 may be connected to the main PCB 82 as illustrated in FIG. 8. The sub PC unit 84 may be connected to the main PCB 82 through a separate cable or a board to board connector.

The sub PCB 84 may be an electrode PCB for electrically connecting the iontophoresis electrode 5.

The cosmetic device may include a spring 86.

The spring 86 may be disposed between the sub PCB 84 and the iontophoresis electrode 5.

The spring 86 may be made of a conductor and may electrically connect the iontophoresis electrode 5 to the sub PCB 84.

An example of spring 84 may be a coil spring.

A contact body 58 that is in contact with the spring 84 may be disposed on the iontophoresis electrode 5. The contact body 58 may be provided to protrude from the hidden body 52 toward the sub PCB 84.

An opening through which the contact body 58 passes may be defined in the second side case 16. An electrode sealing member 59 (see FIG. 8) may be disposed on the second side case 16 to seal a gap between the second side case 16 and the contact body 58. The electrode sealing member 59 may minimize moisture from being penetrated between the second side case 16 and the contact body 58.

An example of the electrode sealing member 59 may be a waterproof O-ring disposed between an outer circumference of the contact body and the second side case 16.

An example of using the cosmetic device is for allowing the user to perform the cleansing by using the brush unit 4 and promote the absorption of the cosmetics, etc. by using the absorption-promoting head 2.

After inputting power through the power input unit 11a, the user may select the cleansing mode through the mode input unit 11b, and when selecting the cleansing mode, the controller 8 may turn on the vibration motor 6 and the ultrasonic vibrator 34.

The user may place the brush unit 4 on the user's skin while wrapping the absorption-promoting head 2 by using the his/her hand, the inner head 33 may transmit the ultrasonic waves and the vibration to the user's skin, and the brush unit 4 may cleanse the user's skin while vibrating.

When the cleaning is completed in the cleaning mode as described above, the user may select the absorption-promoting mode through the mode input unit 11b, and the controller 8 may turn on the head ultrasonic vibrator 9 when selecting the absorption-promoting mode to apply the current to the absorption-promoting head 2 and the iontophoresis electrode 5.

After applying the cosmetics to the skin, the user may be in contact with the iontophoresis electrode 5 while wrapping the brush unit 4 through the his/her hand and may place the absorption-promoting head 2 on the user's skin.

The absorption-promoting head 2 and the iontophoresis electrode 5 may be electrically connected by the user, and the ions may be generated from the absorption-promoting head 2, and then, the ions may push the electric charges in the cosmetics into the skin so that the absorption of the cosmetics into the skin is promoted.

The above-disclosed subject matter is to be considered illustrative, and not restrictive, and the appended claims are intended to cover all such modifications, enhancements, and other embodiments, which fall within the true spirit and scope of the present invention.

Thus, the embodiment of the present invention is to be considered illustrative, and not restrictive, and the technical spirit of the present invention is not limited to the foregoing embodiment.

Therefore, the scope of the present invention is defined not by the detailed description of the invention but by the appended claims, and all differences within the scope will be construed as being included in the present invention.

1. A cosmetic device comprising:
a case in which a space is defined;
an absorption-promoting head disposed on one surface of both side surfaces of the case;
an ultrasonic head and a brush unit, which are disposed on the other surface of both the side surfaces of the case;
a vibration motor accommodated in the space to vibrate at least one of the ultrasonic head or the brush unit;
a controller accommodated in the space; and
an iontophoresis electrode which is closer to the brush unit than to the absorption-promoting head and of which a portion is exposed to the outside of the case.

2. The cosmetic device according to claim 1, wherein the controller is configured to perform:
an absorption-promoting mode in which the absorption-promoting head and the iontophoresis electrode are electrically connected to each other; and
a cleansing mode in which the ultrasonic head and the vibration motor operate.

3. The cosmetic device according to claim 1, wherein the case comprises:
a center case of which an outer surface is concavely recessed;
a first side case coupled to one side of the center case; and
a second case coupled to the other side of the center case,
wherein the second side case has an opening through which the ultrasonic head passes.

4. The cosmetic device according to claim 3, wherein the center case has a horizontal width that gradually increases downward.

5. The cosmetic device according to claim 3, further comprising an inner sealing member disposed along an inner circumferential surface of the center case.

6. The cosmetic device according to claim 3, further comprising:
a first side case sealing member disposed on the first side case to seal a gap between the first side case and the center case; and
a second side case sealing member disposed on the second side case to seal a gap between the second side case and the center case.

7. The cosmetic device according to claim 1, wherein a distance between an upper end of the absorption-promoting head and an upper end of the brush unit is less than a distance between a lower end of the absorption-promoting head and a lower end of the brush head.

8. The cosmetic device according to claim 1, further comprising a head ultrasonic vibrator disposed on the absorption-promoting head,
wherein an inner space is formed in the absorption-promoting head, and the head ultrasonic vibrator is accommodated in the inner space.

9. The cosmetic device according to claim 1, wherein the absorption-promoting head has a cam shape.

10. The cosmetic device according to claim 1, wherein a distance between an upper end of the brush unit and the ultrasonic head is greater than a distance between a lower end of the brush unit and the ultrasonic head.

11. The cosmetic device according to claim 1, wherein the brush head is configured to cover a portion of the iontophoresis electrode outside the case.

12. The cosmetic device according to claim 1, wherein the iontophoresis electrode comprises:
a hidden body hidden by the brush unit between the case and the brush unit; and
an exposed body extending from the hidden body and exposed to the outside of the case.

13. The cosmetic device according to claim 1, wherein the ultrasonic head comprises:
an inner head having an accommodation space therein and disposed inside the brush unit; and
an ultrasonic vibrator accommodated in the accommodation space formed inside of the inner head.

14. The cosmetic device according to claim 13, further comprising an inner head sealing member configured to seal a gap between the inner head and the case, which are disposed on an outer circumference of the inner head.

15. The cosmetic device according to claim 1, wherein the controller comprises:
a main PCB;
a sub PCB connected to the main PCB,
wherein a spring configured to electrically connect the iontophoresis electrode to the sub PCB is disposed between the sub PCB and the iontophoresis electrode.
